(19) United States
(12) Patent Application Publication
(10) Pub. No.: US 2021/0380569 A1
(43) Pub. Date: Dec. 9, 2021

(54) Process for Preparation of Optically Enriched Isoxazolines

(71) Applicant: BASF SE, Ludwigshafen (DE)

(72) Inventors: Roland Goetz, Ludwigshafen (DE); Michael Rack, Ludwigshafen (DE); Martin John McLaughlin, Ludwigshafen (DE); Harish Shinde, Navi Mumbai (IN); Ritesh Karalkar, Navi Mumbai (IN); Kailaskumar Borate, Navi Mumbai (IN); Karsten Koerber, Ludwigshafen (DE); Arun Narine, Ludwigshafen (DE); Nikolas Huwyler, Ludwigshafen (DE)

(21) Appl. No.: 17/288,976

(22) PCT Filed: Oct. 28, 2019
(86) PCT No.: PCT/EP2019/079324

§ 371 Date: Apr. 27, 2021

(30) Foreign Application Priority Data
Nov. 6, 2018 (DE) 18204501.3

(51) Int. Cl. C07D 413/04; B01J 31/02

(52) U.S. Cl. CPC C07D 413/04; B01J 31/0244

(57) ABSTRACT

The invention relates to a process for preparing optically enriched isoxazoline compounds of formula (I), wherein the variables are as defined in the specification, and the shown enantiomer has at least 80% ee; by oxo-Michael addition of hydroxyl amine or its salt to an enone of formula (II), wherein the variables have the meanings given for formula (I), in the presence of a catalyst of formula (III) and a base.

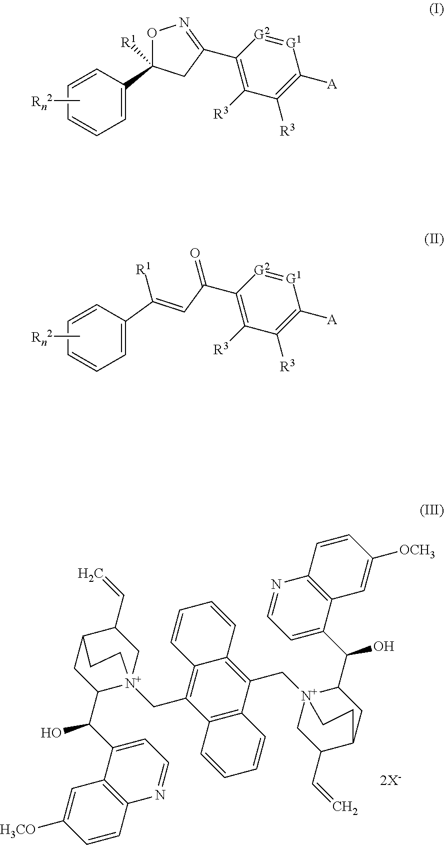

The invention relates to a process for the preparation of optically enriched isoxazoline compounds of formula I

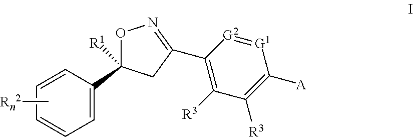

wherein
R¹ is halomethyl;
each R² is independently H, halogen, CN, N₃, NO₂, SCN, SF₅, C₁-C₆-alkyl, C₃-C₈-cycloalkyl, C₂-C₆-alkenyl, C₂-C₆-alkynyl, which groups are unsubstituted, partially or fully halogenated and/or substituted with one or more same or different R⁸,
Si(R¹²)₃, OR⁹, S(O)ₙR⁹, NR^{10a}R^{10b},
phenyl which is unsubstituted or partially or fully substituted with R¹¹, and a 3- to 10-membered saturated, partially or fully unsaturated heteromonocyclic or heterobicyclic ring containing 1, 2, 3 or 4 heteroatoms N, O, and/or S as ring members, which ring is unsubstituted, or substituted with one or more same or different R¹¹, preferably the unsubstituted or substituted heterocycle;
n is 0, 1, or 2;
G¹, G² are each CR³, or together form a sulfur atom;
each R³ is independently selected from the meanings mentioned for R²,
or two R³ bonded to adjacent carbon atoms may form a five- or sixmembered saturated, partially or fully unsaturated carbocyclic ring, or a dihydrofurane, or
R³ bonded to carbon atom in position G¹ form a bond to the chain *-Q-Z- in group A²;
A is a group A¹, A², A³, or A⁴; wherein
   A¹ is C(═W)Y;
   W is O, or S;
   Y is N(R⁵)R⁶, or OR⁹;
   A² is

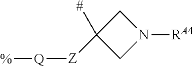

wherein # denotes the bond of group A, and % denotes the bond to G¹;
Q-Z is % —CH₂—O—*, ‘% —CH₂—S(O)ₙ—*, or % —C(═O)—O—*, wherein % marks the bond of Q to phenyl, and * the bond of Z to azetidin; and
R^{A4} is H or C(═O)R^{4A}, wherein
   R^{4A} is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₄-alkylcarbonyl, which aliphatic groups are unsubstituted or substituted with one or more radicals R⁴¹;
   C₃-C₆-cycloalkyl, C₃-C₆-halocycloalkyl which cyclic groups are unsubstituted or substituted with one or more R⁴²;
   C(═O)N(R⁴³)R⁴⁴, N(R⁴³)R⁴⁵, CH═NOR⁴⁶;
   phenyl, heterocycle, or hetaryl which rings are unsubstituted or partially or fully substituted with R^{A};
   R⁴¹ is independently OH, CN, C₁-C₆-alkoxy, C₁-C₆-haloalkoxy, S(O)ₙ—C₁-C₆-alkyl, S(O),-C₁-C₆-haloalkyl, C(═O)N(R9R⁴⁴,
   C₃-C₆-cycloalkyl, or C₃-C₆-halocycloalkyl which cycles are unsubstitued or substituted with one or more R⁴¹¹; or
   phenyl, heterocycle or hetaryl which rings are unsubstitued or partially or fully substituted with R^{A};
   R⁴¹¹ is independently OH, CN, C₁-C₂-alkyl, or C₁-C₂-haloalkyl;
   R⁴³ is H, or C₁-C₆-alkyl,
   R⁴⁴ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted with a cyano;
   R⁴⁵ H, C₁-C₆-alkyl, C₁-C₆-haloalkyl, C₂-C₄-alkenyl, C₂-C₄-alkynyl, CH₂-CN, C₃-C₆-cycloalkyl, C₃-C₆-halocycloalkyl, C₃-C₆-cycloalkylmethyl, C₃-C₆-halocycloalkylmethyl, phenyl and hetaryl which aromatic rings are unsubstituted or partially or fully substituted with R^{A};
   R⁴² C₁-C₆-alkyl, C₁-C₆-haloalkyl, or a group as defined for R⁴¹;
   R⁴⁶ is independently H, C₁-C₆-alkyl, or C₁-C₆-haloalkyl;
   R^{A} is independently selected from halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₙ—C₁-C₄-alkyl, S(O)ₙ—C₁-C₄-haloalkyl, C₁-C₄-alkylcarbonyl, C₁-C₄-haloalkylcarbonyl, C(═O)N(R⁴³)R⁴⁴; or
   two R^{A} present on the same carbon atom of a saturated or partially saturated ring may form together ═O or ═S; or
   two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
A³ is CH₂—NR⁵C(═W)R⁶;
A⁴ is halogen, or cyano;
R⁵ is independently selected from the meanings mentioned for R²;
R⁶ is H, CN, C₁-C₁₀-alkyl, C₃-C₈-cycloalkyl, C₂-C₁₀-alkenyl, C₂-C₁₀-alkynyl, which groups are unsubstituted, partially or fully halogenated and/or substituted with one or more same or different R⁸; or
S(O)ₙ—R⁹, or C(═O)R⁸; or
a 3- to 8-membered saturated, partially or fully unsaturated heterocyclic ring, which ring may contain 1, 2, 3, or 4 heteroatoms O, S, N, C═O and/or C═S as ring members, which heterocyclic ring is unsubstituted or partially or fully substituted with same or different halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R⁸, or phenyl which may be partially or fully substituted with R¹¹;
or R⁵ and R⁶ together form a group ═C(R⁸)₂, ═S(O)ₘ(R⁹)₂, ═NR^{10a}, or ═NOR⁹;
R^{7a}, R^{7b} are each independently H, halogen, CN, C₁-C₆-alkyl, C₃-C₈-cycloalkyl, C₂-C₆-alkenyl, or C₂-C₆-alkynyl, which groups are unsubstituted, partially or fully halogenated and/or substituted with same or different R⁸;
each R⁸ is independently CN, N₃, NO₂, SCN, SF₅, C₃-C₈-cycloalkyl, C₃-C₈-halocycloalkyl, wherein the carbon chains may be substituted with one or more R¹³;
Si(R¹²)₃, OR⁹, OSO₂R⁹, S(O)ₙR⁹, N(R^{10a})R^{10b}, C(═O)N(R^{10a})R^{10b}, C(═S)N(R^{10a})R^{10b}, C(═O)OR⁹, CH═NOR⁹,
phenyl, which is unsubstituted or partially or fully substituted with same or different R¹⁶, or
a 3-, 4-, 5-, 6- or 7-membered saturated, partially or fully unsaturated heterocyclic ring comprising 1, 2 or 3 heteroatoms N, O, and/or S as ring members, which ring is unsubstituted or partially or fully substituted with same or different R¹⁶, or
two R⁸ present on the same carbon atom of an alkyl, alkenyl, alkynyl or cycloalkyl group together form a group ═O, ═C(R¹³)₂; ═S; ═S(O)ₘ(R¹⁵)₂,
═S(O)ₘR¹⁵N(R^{14a})R^{14b}, ═NR^{10a}, ═NOR⁹; or ═NN(R^{10a})R^{10b}; or
two radicals R⁸, together with the carbon atoms of the alkyl, alkenyl, alkynyl or cycloalkyl group which they are bonded to, form a 3-, 4-, 5-, 6-, 7- or 8-membered saturated or partially unsaturated carbocyclic or heterocyclic ring, which heterocyclic ring comprises 1, 2, 3 or 4 heteroatoms N, O, and/or S as ring members, and which ring is unsubstituted, or partially or fully substituted with same or different R¹⁶; and
R⁸ as a substituent on a cycloalkyl ring may additionally be C₁-C₆-alkyl, C₁-C₆-haloalkyl, C₂-C₆-alkenyl, C₂-C₆-haloalkenyl, C₂-C₆-alkynyl, and C₂-C₆-haloalkynyl, which groups are unsubstituted, or partially or fully substituted with same or different R¹³; and
R⁸ in the groups C(═O)R⁸ and ═C(R⁸)₂ may additionally be H, halogen, C₁-C₆-alkyl, C₁-C₆-haloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³;
each R⁹ is independently H, CN, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³, or
C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b},
C₁-C₆-alkyl-C(═NR¹⁴)N(R^{14a})R^{14b}, Si(R¹²)₃, S(O)ₙR¹⁵, S(O)ₙN(R^{14a})R^{14b}, N(R^{10a})R^{10b}, N═C(R¹³)₂, C(═O)R¹³, C(═O)N(R^{14a})R^{14b}, C(═S)N(R^{14a})R^{14b}, C(═O)OR¹⁵, or
phenyl, which is unsubstituted, or partially or fully substituted with R¹⁶; and
a 3- to 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; and
R⁹ in the groups S(O)ₙR⁹ and OSO₂R⁹ may additionally be C₁-C₆-alkoxy, or C₁-C₆-haloalkoxy;
R^{10a}, R^{10b} are independently from one another H, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R¹³;
C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b},
C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, S(O)ₙR¹⁵, S(O)ₙN(R^{14a})R^{14b}, C(═O)R¹³, C(═O)OR¹⁵,
C(═O)N(R^{14a})R^{14b},
C(═S)R¹³, C(═S)SR¹⁵, C(═S)N(R^{14a})R^{14b}, C(NR¹⁴)R¹³;
phenyl, which is unsubstituted, or partially or fully substituted with same or different R¹⁶; and
a 3-, 4-, 5-, 6- or 7-membered saturated, partially or fully unsaturated heterocyclic ring comprising 1, 2, 3 or 4 heteroatoms N, O, and/or S as ring members, which ring is unsubstituted, or partially or fully substituted with same or different R¹⁶, preferably unsubstituted or substituted hetaryl; or
R^{10a} and R^{10b} together with the nitrogen atom they are bonded to form a 3- to 8-membered saturated, partially or fully unsaturated heterocyclic ring, which ring may additionally contain one or two heteroatoms N, O, and/or S as ring members, which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with R¹⁶, and a 3-, 4-, 5-, 6,- or 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; or
R^{10a} and R^{10b} together form a group ═C(R¹³)₂, ═S(O)ₘ(R¹⁵)₂, ═S(O)ₘR¹⁵N(R^{14a})R^{14b}, ═NR¹⁴, or ═NOR¹⁵;
R¹¹ is halogen, CN, N₃, NO₂, SCN, SF₅, C₁-C₁₀-alkyl, C₃-C₈-cycloalkyl, C₂-C₁₀-alkenyl, C₂-C₁₀-alkynyl, which groups are unsubstituted, partially or fully halogenated, and/or may be substituted with same or different R⁸, or
OR⁹, NR^{10a}R^{10b}, S(O)ₙR⁹, Si(R¹²)₃;
phenyl, which is unsubstituted, or partially or fully substituted with same or different R¹⁶; and
a 3- to 7-membered saturated, partially or fully unsaturated aromatic heterocyclic ring comprising 1, 2, 3, or 4 heteroatoms N, O, and/or S as ring members, which ring is unsubstituted, or partially or fully substituted with same or different R¹⁶; or
two R¹¹ present on the same ring carbon atom of an unsaturated or partially unsaturated heterocyclic ring may together form a group ═O, ═C(R¹³)₂, ═S,
═S(O)ₘ(R¹⁵)₂, ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3- to 9-membered ring, which ring may contain 1 or 2 heteroatoms O, S, N, and/or NR¹⁴, and/or 1 or 2 groups C═O, C═S, C═NR¹⁴ as ring members, and which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with same or different R¹⁶, and a 3- to 7-membered saturated, partially or fully unsaturated heterocyclic ring containing 1, 2, or 3 heteroatoms N, O, and/or S as ring members, which ring is unsubstituted, or partially or fully substituted with same or different R¹⁶;
each R¹² is independently C₁-C₄-alkyl and phenyl, which is unsubstituted, or partially or fully substituted with same or different C₁-C₄-alkyl;
each R¹³ is independently CN, NO₂, OH, SH, SCN, SF₅, C₁-C₆-alkoxy, C₁-C₆-haloalkoxy, SOₙ-C₁-C₆-alkyl, SOₙ-C₁-C₆-haloalkyl, Si(R¹²)₃, —C(═O)N(R^{14a})R^{14b},
C₃-C₈-cycloalkyl which is unsubstituted, partially or fully halogenated or substituted with 1 or 2 same or different C₁-C₄-alkyl, C₃-C₄-cycloalkyl, C₁-C₄-alkoxy, C₁-C₄-haloalkoxy and/or oxo; phenyl, benzyl, phenoxy, where the phenyl moiety may be substituted with one or more same or different R¹⁶; and a 3- to 7-membered saturated, partially or fully unsaturated heterocyclic ring containing 1, 2, or 3 heteroatoms N, O, and/or S, as ring members, which ring is unsubstituted, or partially or fully substituted with same or different R¹⁶; or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl); and
R¹³ as a substituent of a cycloalkyl ring may additionally be C₁-C₆-alkyl, C₂-C₆-alkenyl or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo; and
R¹³ in groups ═C(R¹³)₂, N═C(R¹³)₂, C(═O)R¹³, C(═S)R¹³, and C(═NR¹⁴)R¹³ may additionally be H, halogen, C₁-C₆-alkyl, C₂-C₆-alkenyl, or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo;
each R¹⁴ is independently H, CN, C₁-C₆-alkoxy, C₁-C₆-haloalkoxy, SOₙ-C₁-C₆-alkyl, SOₙ-C₁-C₆-haloalkyl, Si(R¹²)₃;
C₁-C₆-alkyl, C₂-C₆-alkenyl, C₂-C₆-alkynyl, which groups are unsubstituted, partially or fully halogenated, or substituted with 1 or 2 CN, C₁-C₄-alkoxy, C₁-C₄-haloalkoxy, SOₙ-C₁-C₄-alkyl, C₃-C₆-cycloalkyl which is unsubstituted or substituted with 1 or 2 substituents halogen and CN;
and oxo;
C₃-C₈-cycloalkyl which is unsubstituted, or partially or fully halogenated or substituted with 1 or 2 CN, C₁-C₄-alkyl, C₁-C₄-alkoxy, C₁-C₄-haloalkoxy, SOₙ-C₁-C₆-alkyl, C₃-C₄-cycloalkyl, C₃-C₄-cycloalkyl-C₁-C₄-alkyl-, which groups are unsubstituted, or substituted with 1 or 2 substituents selected from halogen and CN;
phenyl, benzyl, pyridyl, phenoxy, which cyclic moieties are unsubstituted, or substituted with one or more same or different halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and C₁-C₆-alkoxycarbonyl; and a 3-, 4-, 5- or 6-membered saturated, partially or fully unsaturated heterocyclic ring comprising 1, 2 or 3 heteroatoms N, O, and/or S as ring members, which ring is unsubstituted, or partially or fully substituted with same or different R¹⁶;
R^{14a} and R^{14b} independently of each other, have one of the meanings given for R¹⁴; or
R^{14a} and R^{14b}, together with the nitrogen atom to which they are bound, form a 3- to 7-membered saturated, partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy; or
R^{14a} and R¹⁴ or R^{14b} and R¹⁴, together with the nitrogen atoms to which they are bound in the group C(═NR¹⁴)N(R^{14a})R^{14b}, form a 3- to 7-membered partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-haloalkyl, C₁-C₄-alkoxy, or C₁-C₄-haloalkoxy;
each R¹⁵ is independently H, CN, Si(R¹²)₃
C₁-C₆-alkyl, C₂-C₆-alkenyl, C₂-C₆-alkynyl, which groups are unsubstituted, partially or fully halogenated, or substituted with 1 or 2 radicals C₃-C₄-cycloalkyl, C₁-C₄-alkoxy, C₁-C₄-haloalkoxy, SOₙ-C₁-C₆-alkyl, or oxo;
C₃-C₈-cycloalkyl which is unsubstituted, partially or fully halogenated or substituted with 1 or 2 radicals C₁-C₄-alkyl, C₃-C₄-cycloalkyl, C₁-C₄-alkoxy, C₁-C₄-haloalkoxy, SOₙ-C₁-C₆-alkyl, or oxo;
phenyl, benzyl, pyridyl, and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl;
each R¹⁶ is independently halogen, NO₂, CN, OH, SH, C₁-C₆-alkoxy, C₁-C₆-haloalkoxy, SOₙ-C₁-C₆-alkyl, SOₙ-C₁-C₆-haloalkyl, C₁-C₄-alkylcarbonyl, C₁-C₄-haloalkylcarbonyl, C₁-C₄-alkoxycarbonyl, C₁-C₄-haloalkoxycarbonyl, aminocarbonyl, C₁-C₄-alkylaminocarbonyl, di-(C₁-C₄-alkyl)-aminocarbonyl, Si(R¹²)₃;
C₁-C₆-alkyl, C₂-C₆-alkenyl, C₂-C₆-alkynyl, which groups are unsubstituted, partially or fully halogenated, or substituted with 1 or 2 radicals CN, C₃-C₄-cycloalkyl, C₁-C₄-alkoxy, C₁-C₄-haloalkoxy, or oxo;
C₃-C₈-cycloalkyl which is unsubstituted, partially or fully halogenated or substituted with 1 or 2 radicals CN, C₁-C₄-alkyl, C₃-C₄-cycloalkyl, C₁-C₄-alkoxy, C₁-C₄-haloalkoxy, or oxo;
phenyl, benzyl, pyridyl and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl; or
two R¹⁶ present together on the same atom of an unsaturated or partially unsaturated ring may be ═O, ═S, ═N(C₁-C₆-alkyl), ═NO-C₁-C₆-alkyl, ═CH(C₁-C₄-alkyl), or ═C(C₁-C₄-alkyl)₂; or
two R¹⁶ on two adjacent carbon atoms form together with the carbon atoms they are bonded to a 4- to 8-membered saturated, partially or fully unsaturated ring, wherein the ring may contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy;
each n is independently 0, 1, or 2; and
each m is independently 0, or 1;

wherein the shown enantiomer has at least 80% ee;

by oxo-Michael addition of hydroxyl amine or its salt to an enone of formula II,

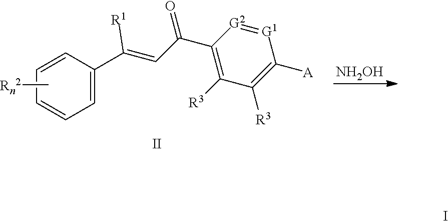

wherein the variables have the meanings given for formula I, in the presence of a catalyst of formula III

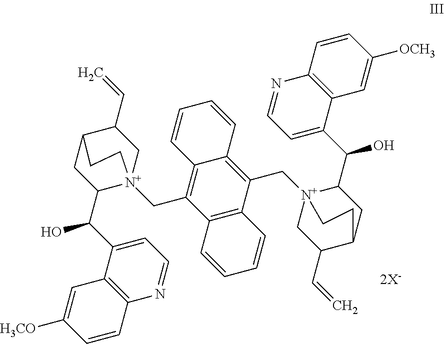

wherein X is a counteranion;

and a base.

The isoxazoline active compounds I wherein group A is A¹, A², or A³, and their pesticidal activity are generally known from WO 2005/085216, WO 2007/026965, WO 2009/00289, WO 2011/067272, WO 2012/120399, WO 2014/090918, WO 2016/102482, and WO 2018/197466. Compounds of formula I with group A⁴ are valuable intermediates for the synthesis of formula I active compounds.

WO 2009/063910, WO 2012/156400, WO 2013/069731, WO 2014/79937, and WO 2014/79941 describe asymmetric syntheses of some isooxazoline compounds of formula I by using cinchona alkaloid-based phase-transfer catalysts. The processes require relatively high catalyst loadings and yield enantiomeric excesses of formula I compounds which still leave room for improvement.

Objective task for the invention therefore is providing an economical, industrially applicable manufacturing process for optically enriched compounds of formula I. This task is achieved by the process defined in the outset. The presence of a catalyst III as defined herein in the reaction of compound II ensures a quick and complete transformation at moderate temperatures.

Formula III catalyst is described in the art for enantioselective Michael addition reactions of cyclic esters with Michael acceptor to form C—C bonds (cf. Tetrahedron: Asymmetry 2009, 20, 2651-2654; Tetrahedron: Asymmetry 2010, 21, 2872-2878; Tetrahedron: Asymmetry 2012, 23, 176-180).

In the invention this catalyst is used in asymmetric Oxa-Michael addition of hydroxyl amine with an enone to form an enantioselective C—O bond. The process yields formula I compounds in good yield with at least 80% ee by using low catalyst loadings.

The reaction of an enone of formula II, wherein the variables have the meanings given in the outset, with hydroxyl amine or its salt is usually carried out at temperatures of from −30° C. to 35° C., preferably from −10° C. to 0° C., in an inert solvent, in the presence of catalyst of formula III. The formula III catalyst is known from Tetrahedron: Asymmetry 2009, 20,2651-2654.

Suitable solvents are preferably water immiscible solvents, such as aliphatic hydrocarbons such as pentane, hexane, cyclohexane, and petrol ether, aromatic hydrocarbons such as toluene, o-, m-, and p-xylene, halogenated hydrocarbons such as methylene chloride, dichloroethane, and chloroform, ethers such as diethylether, diisopropylether, tert.-butyl-methylether, anisole, and ketones such as methyl ethyl ketone, diethyl ketone, and tert.-butyl methyl ketone, alcohols such as, n-propanol, n-butanol, preferably halogenated hydrocarbons such as methylene chloride, dichloroethane, and chloroform. It is also possible to use mixtures of the solvents mentioned.

Suitable bases are in general, inorganic compounds, such as alkali metal and alkaline earth metal hydroxides, such as LiOH, NaOH, KOH and Ca(OH)₂, alkali metal and alkaline earth metal oxides, such as Li₂O, Na₂O, CaO, and MgO, and alkaline earth metal carbonates, such as Li₂CO₃, Na₂CO₃, K₂CO₃ and CaCO₃, and also alkali metal bicarbonates, such as NaHCO₃, moreover organic bases, e.g. tertiary amines, such as trimethylamine, triethylamine (NEt₃), diisopropylethylamine and N-methylpiperidine, pyridine, substituted pyridines, such as collidine, lutidine and 4-dimethylaminopyridine, and also bicyclic amines, such as DBU (1,8-Diazabicyclo(5.4.0)undec-7-ene) and DBN (1,5-Diazabicyclo[4.3.0]non-5-ene). Particular preference is given to alkali metal and alkaline earth metal hydroxides, such as LiOH, NaOH, KOH, and Ca(OH)₂, such as NaOH, and KOH.

The bases are generally employed in catalytic amounts; however, they can also be used in equimolar amounts or in excess. Under certain conditions an excess up to 10 mol equivalents of compound II may be advantageous.

For practical reasons hydroxylamine is preferably used in the form of an aqueous solution, alternatively as acid addition salt, such as halogenide or sulfate, preferably halogenide, particularly as HCl addition salt.

Hydroxylamine is generally employed in equimolar amounts; however, it can also be used in excess. Under certain conditions an excess up to 10 mol equivalents of compound II may be advantageous.

The catalyst III is used in 0.01 to 0.5, preferably 0.01 to 0.2, particularly about 0.02 to 0.1 mol equivalents of compound II. The starting materials are generally reacted with one another in equimolar amounts. In terms of yield, it may be advantageous to employ an excess of hydroxyl amine, based on II.

Starting materials of formula II required for preparing the compounds I are commercially available or known from the literature or can be prepared as outlined above, or in accordance with the literature cited.

In case group A in formula I is different from group A in the envisaged final isooxazoline active compound the cyclisation as described in the outset yields in an intermediate compound of formula Ia, which corresponds to formula I. The intermediate Ia is transformed to the active compound in a subsequent reaction step.

If in compounds la group A is A¹ or A³ different from group A in the envisaged final active compounds I, the process also comprises the amidation of Ia with an appropriate amine IV under conditions known in the art, e.g. WO2004/22536.

In case in formula II group A is A¹ which is COOR⁹ or CON(R⁵)R⁶, wherein R⁵ and R⁶ are as defined for formula I, and preferably are H or C₁-C₆-alkyl, and R⁹ is H or a leaving group, the reaction yields intermediate compounds Ia′. Compounds of formula I can be prepared by reacting carboxylic acids or acid derivatives of formula Ia′ with an amine of formula IV in an amidation reaction.

In formula Ia′ the variables are as defined for formula I, and A is A¹ C(O)Y, wherein
Y is OR⁹, wherein R⁹ is H or preferably C₁-C₆-alkyl, such as CH₃ or C₂H₅, or
Y is N(R⁵)R⁶, wherein R⁵ and R⁶ are preferably H or C₁-C₆-alkyl.

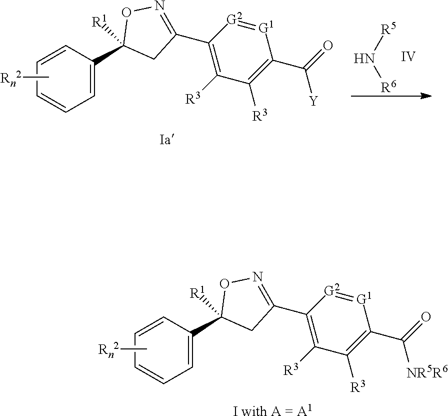

The amidation reaction is preferably carried out by direct reaction with the amine IV, or by prior transformation of carboxylic acids of formula Ia′ (compounds of formula Ia with Y being OH) with oxalyl chloride [(COCl)₂] or thionylchloride (SOCl₂) to the corresponding acid chlorides of formula Ib, followed by reaction with an amine of formula IV. The reaction is preferably carried out in the presence of an organic base such as, NEt₃, N-ethyl-N,N-diisopropylamine, pyridine, or substituted pyridines such as collidine or lutidine. Optionally a nucleophilic catalyst such as 4-(N,N-dimethylamino)pyridine (“DMAP”) can be employed in the reaction. Suitable solvents are halogenated hydrocarbons such as, dichloromethane, chloroform, and chlorobenzene, or polar aprotic solvents such as THF, 1,4-dioxane, and N,N-dimethylformamide (DMF), or aromatic hydrocarbons such as benzene, toluene, o-, m-, and p-xylene, or mixtures thereof. The transformation is usually carried out at temperatures from −40° C. to 100° C., preferably from 0° C. to 30° C. The starting materials are generally reacted with one another in equimolar amounts. In terms of yield, it may be advantageous to employ an excess of IV, based on Ia.

Compounds of formula Ia′, or formula I compounds with A being A¹ can be obtained from compound wherein A is A⁴ being halogen, such as bromine or iodine (formula Id).

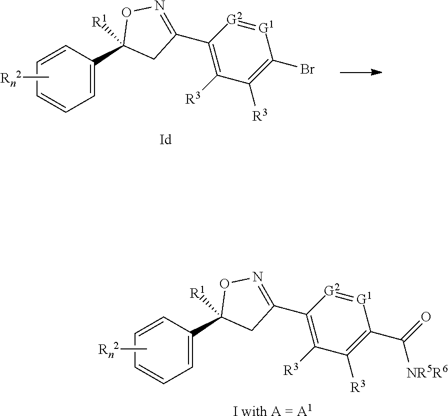

This transformation is usually carried out at temperatures of from 50° C. to 115° C., preferably from 75° C. to 110° C., in an inert solvent, in the presence of a base and a catalyst [cf. WO 2012/059441].

Compounds of formula I with A being A³ can preferably be prepared by reduction of nitrils of formula Ia wherein A is A⁴ being cyano (formula Ia″) to the corresponding amine of formula Ic, and subsequent acylation of Ic with a carboxylic acid derivative of formula V. In formula Ia″ the variables are as defined for formula I.

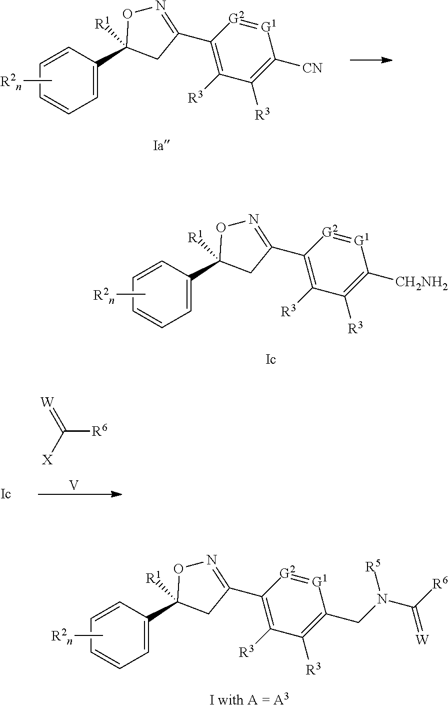

The reduction of Ia″ to Ic is usually carried out at temperatures of from −10° C. to +110° C., preferably from 0° C. to +60° C., in an inert solvent, in the presence of a base, a reducing agent and a catalyst [cf. JP 2010235590].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, TBME, dioxane, anisole, and THF, nitrils such as acetonitrile, and propionitrile, alcohols such as methanol, ethanol, n-propanol, isopropanol, n-butanol, and tert.-butanol, moreover water; preferably alcohols, ethers, and water. It is also possible to use mixtures of the solvents mentioned.

Suitable bases are, in general, inorganic compounds, such as alkali metal and alkaline earth metal hydroxides, such as LiOH, NaOH, KOH, and Ca(OH)₂, alkali metal and alkaline earth metal oxides, such as Li₂O, Na₂O, CaO, and MgO, alkali metal and alkaline earth metal hydrides, such as LiH, NaH, KH, and CaH₂, alkali metal and alkaline earth metal carbonates, such as Li₂CO₃, Na₂CO₃, K₂CO₃ and CaCO₃, and also alkali metal bicarbonates, such as NaHCO₃, moreover organic bases, e.g. tertiary amines, such as trimethylamine, NEt₃, diisopropylethylamine and N-methylpiperidine, pyridine, substituted pyridines, such as collidine, lutidine and 4-dimethylaminopyridine, and also bicyclic amines, such as DBU and DBN. Particular preference is given to alkali metal and alkaline earth metal carbonates and alkali metal bicarbonates, such as NaHCO₃.

The bases are generally employed in catalytic amounts; however, they can also be used in equimolar amounts or in excess.

Suitable catalysts are nickel carbonyl, Raney nickel or nickel dichloride.

Suitable reducing agents are hydrogen gas, or alkali metal hydrides such as sodium borohydride or lithium borohydride.

The starting materials are generally reacted with one another in equimolar amounts. In terms of yield, it may be advantageous to employ an excess of V, based on Ic.

The acylation is usually carried out at temperatures of from −10° C. to 110° C., preferably from 0° C. to 60° C., in an inert solvent, in the presence of a base and a catalyst [cf. Organic Letters, 18(23), 5998-6001, 2016].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, TBME, dioxane, anisole, and THF, nitrils such as acetonitrile, and propionitrile, alcohols such as methanol, ethanol, n-propanol, isopropanol, n-butanol, and tert.-butanol, moreover water; preferably halogenated hydrocarbons and aromatic hydrocarbons. It is also possible to use mixtures of the solvents mentioned.

Suitable bases are, in general, inorganic compounds, such as alkali metal and alkaline earth metal hydroxides, such as LiOH, NaOH, KOH and Ca(OH)₂, alkali metal and alkaline earth metal oxides, such as Li₂O, Na₂O, CaO, and MgO, alkali metal and alkaline earth metal hydrides, such as LiH, NaH, KH, and CaH₂, alkali metal and alkaline earth metal carbonates, such as Li₂CO₃, Na₂CO₃, K₂CO₃ and CaCO₃, and also alkali metal bicarbonates, such as NaHCO₃, moreover organic bases, e.g. tertiary amines such as trimethylamine, triethylamine, diisopropylethylamine and N-methylpiperidine, pyridine, substituted pyridines such as collidine, lutidine and 4-dimethylaminopyridine, and also bicyclic amines. Particular preference is given to alkali metal and alkaline earth metal carbonates and alkali metal bicarbonates, such as

NaHCO₃. The bases are generally employed in catalytic amounts; however, they can also be used in equimolar amounts, in excess or, if appropriate, as solvent.

Suitable catalysts are e.g. 4-N,N-dimethyl aminopyridine, DBU (1,8-Diazabicyclo(5.4.0)un-dec-7-ene), pyridine, DBN; catalytic NaI, KI, LI to activate acid chloride to acid iodide.

The starting materials are generally reacted with one another in equimolar amounts. In terms of yield, it may be advantageous to employ an excess of V, based on Ic.

The reaction mixtures are worked up in a customary manner, for example by mixing with water, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

Furthermore, in one embodiment the invention relates to a process for the manufacture of compounds of formula I comprising the steps of reacting formula II with hydroxy amine or its salt, and amidation Ia′ to the final active compounds I.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (“Me”), ethyl (“Et”), n-propyl, iso-propyl, n-butyl, 2-butyl, iso-butyl, tert-butyl (“^{t}Bu”), n-pentyl, and n-hexyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms.

The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 5- or 6-membered, in particular 6-membered monocyclic heterocyclic non-aromatic radicals. The heterocyclic non-aromatic radicals usually comprise 1, 2 or 3 heteroatoms selected from N, O and S as ring members, wherein S-atoms as ring members may be present as S, SO or SO₂.

The term “hetaryl” includes monocyclic 5- or 6-membered heteroaromatic radicals comprising as ring members 1, 2, or 3 heteroatoms selected from N, O and S.

With respect to the variables, the particularly preferred embodiments of the intermediates correspond to those of the compounds of the formula I.

In a particular embodiment, the variables of the compounds of the formula I have the following meanings, these meanings, both on their own and in combination with one another, being particular embodiments of the compounds of formula I.

The process is particularly suitable for compounds II wherein A is selected from A¹, A², and A³.

In the compounds of the inventive process R¹ is preferably fluoromethyl, in particular CF₃.

The phenyl ring in formula I and its sub formulae, bearing the R²ₙ substitution is preferably a group P

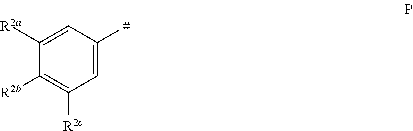

R^{2a} is preferably selected from F, Cl, Br, CF₃, and OCF₃.

R^{2b} and R^{2c} are independently preferably selected from H, F, Cl, Br, CF₃, and OCF₃.

Particularly preferred is each one of the following combinations of R^{2a}, R^{2b}, and R^{2c} wherein each line of Table A denotes a substitution pattern of the phenyl ring P bearing the R^{2a}, R^{2b}, and R^{2c} moieties.

TABLE A

| No. | R^{2a} | R^{2b} | R^{2c} |
|---|---|---|---|
| A-1 | F | F | H |
| A-2 | F | H | F |
| A-3 | F | F | F |
| A-4 | F | Cl | F |
| A-5 | F | Br | F |
| A-6 | F | H | Cl |
| A-7 | F | H | Br |
| A-8 | Cl | F | H |
| A-9 | Cl | H | Cl |
| A-10 | Cl | Cl | Cl |
| A-11 | Cl | F | Cl |
| A-12 | Cl | Br | Cl |
| A-13 | Cl | H | Br |
| A-14 | Br | F | H |
| A-15 | Br | H | Br |
| A-16 | Br | F | Br |
| A-17 | Br | Cl | Br |
| A-18 | CF₃ | H | H |
| A-19 | CF₃ | H | F |
| A-20 | CF₃ | H | Cl |
| A-21 | CF₃ | H | Br |
| A-22 | CF₃ | H | CF₃ |
| A-23 | CF₃ | F | F |
| A-24 | CF₃ | F | Cl |
| A-25 | CF₃ | Cl | Cl |
| A-26 | CF₃ | F | H |
| A-27 | OCF₃ | H | F |
| A-28 | OCF₃ | H | Cl |
| A-29 | OCF₃ | F | H |
| A-30 | OCF₃ | H | CF₃ |
| A-31 | OCF₃ | H | H |

Groups A-8, A-9, and A-11 are more preferred patterns in formula I and its sub formulae compounds. A-11 is particularly preferred.

R³ is preferably H, halogen, or CH₃.

In a preferred embodiment G¹ and G² represent each CR³, particularly G¹ is CH and G² is C—Cl, or C—CH₃.

In another embodiment G¹ and G² represent each CR³, wherein the two R³ form a five- or sixmembered saturated carbocyclic ring, or a dihydrofurane.

In another embodiment G¹ and G² together form a sulfur atom.

A preferred embodiment relates to the process for obtaining compounds I wherein A is A¹.

The catalyst III is used preferably in an amount of 0.1-100 mol %, more preferred in 0.5-50 mol %, particularly in 1-20 mol % relative to formula II compounds.

The nature of the counteranion X⁻ in formula III catalyst is of minor importance. For practical reasons it is usually selected from halogen (preferably Cl, Br), BF₄, PF₆, C₁-C₁₀-alkylsulfonate, benzenesulfonate, or methylbenzenesulfonate. Particularly preferred III is used as dibromide.

The processes for obtaining compounds I wherein A is A¹ start preferably from compounds of formula II wherein A is C(═O)Y, and Y is OR⁹, preferably OH, or C₁-C₄-alkoxy, or NR⁵R⁶, wherein R⁵ and R⁶ are H or C₁-C₄-alkyl, preferably Y is NH₂ or NHCH₃. Particularly preferred A group in compounds I and its intermediates is an C₁-C₄-alkylester, such as C(═O)OCH₃.

In A¹ the variables R⁵ and R⁶ have preferably following meanings:
R⁵ is preferably H, C₁-C₄-alkyl;
R⁶ is preferably H, C₁-C₆-alkyl, C₂-C₆-alkenyl, which groups are substituted with one or more same or different R⁸, wherein
R⁸ is preferably C₃-C₈-cycloalkyl, C₃-C₈-halocycloalkyl, wherein the carbon chains may be substituted with one or more R¹³;
S(O)ₙR⁹, N(R^{10a})R^{10b}, C(═O)N(R^{10a})R^{10b}, C(═S)N(R^{10a})R^{10b}, C(═O)OR⁹, CH═NOR⁹,
phenyl, which is unsubstituted or partially or fully substituted with same or different R¹⁶, or
a 3-, 4-, 5-, 6- or 7-membered saturated, partially or fully unsaturated heterocyclic ring comprising 1, 2 or 3 heteroatoms N, O, and/or S as ring members, which ring is unsubstituted or partially or fully substituted with same or different R¹⁶, or
a 5-membered saturated heteromonocyclic ring containing 1, or 2 heteroatoms N, O, and/or S as ring members, which ring is unsubstituted, or substituted with one or more same or different R¹¹, preferably the unsubstituted or substituted HET;
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
R⁹ is preferably H, CN, C₁-C₆-alkyl, C₁-C₆-haloalkyl;
R¹¹ C₁-C₁₀-alkyl, which is unsubstituted, partially or fully halogenated, and/or may be substituted with same or different R⁸, or
OR⁹, NR^{10a}R^{10b}, S(O)ₙR⁹;
two R¹¹ present on the same ring carbon atom of an unsaturated or partially unsaturated heterocyclic ring may together form a group ═O, ═C(R¹³)₂, ═S, ═S(O)ₘ(R¹⁵)₂, ═S(O)ₘR¹⁵N(R^{14a})R^{14b}, ═NR¹⁴, ═NOR¹⁵, or ═NN(R^{14a})R^{14b}.

Another embodiment relates to the process for obtaining compounds I wherein A is A², preferably wherein Q-Z is % —CH₂—O—*, and R⁴ is C₁-C₄-alkylcarbonyl wherein the terminal C-atom of the alkyl is substituted with S(O)ₙ-C₁-C₄-alkyl.

Another embodiment relates to the process for obtaining compounds I wherein A is A³, preferably CH₂—NR⁵C(═O)R⁶, wherein R⁵ is H or CH₃, and R⁶ is H, C₁-C₆-alkyl, C₂-C₆-alkenyl, which groups are substituted with one or more same or different R⁸, wherein R⁸ is as defined and preferred above.

Compounds I and its sub formulae wherein A is A⁴ are intermediates in the inventive process.

Compounds wherein A is A⁴ are preferred intermediates. In one embodiment A⁴ is cyano. In another embodiment A⁴ is halogen, preferably Br, or I.

The process is particularly suitable for synthesis of following active compounds of formula I, which correspond to formulae I.A, and I.B, resp., wherein the variables are as defined and preferred above:

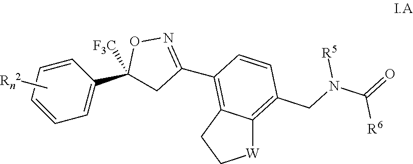

wherein W is CH or O; and

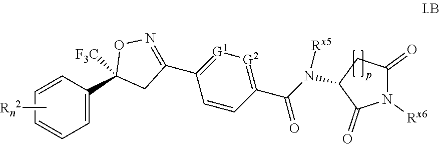

wherein p is 1 or 2; R^{x5} is H or CH₃, and R^{x6} is C₁-C₆-alkyl, C₁-C₄-haloalkyl, C₃-C₆-alkenyl, C₃-C₆-alkynyl, which groups may be substituted with C(═O)OR^{a1}, C(═O)N(R^{a2})R^{a3}, CH═NOR^{a1}, and phenyl, benzyl, which rings are unsubstituted or substituted with halogen, C₁-C₄-alkyl, or C₁-C₄-haloalkyl; wherein R^{a1} is C₁-C₆-alkyl, R^{a2} and R^{a3} are each H or C₁-C₆-alkyl, C₁-C₆-haloalkyl, C₂-C₄-alkenyl, C₂-C₄-alkynyl;

preferably R^{x6} is CH₃, C₂H₅, CH₂(CH₃)₂, CH₂CH═CH₂, CH₂CF₃, CH₂CH₂CF₃, CH₂C₆H₅, or CH₂C(═O)OCH₃.

The process is furthermore particularly suitable for synthesis of following active compounds 1.1, 1.2, 1.3, 1.4, 1.5, and 1.6 of formula I which are known in the art (cf. WO 2011067272; WO 2005085216; WO 200900289; WO 2014090918; WO 2007026965; WO 2012120399):

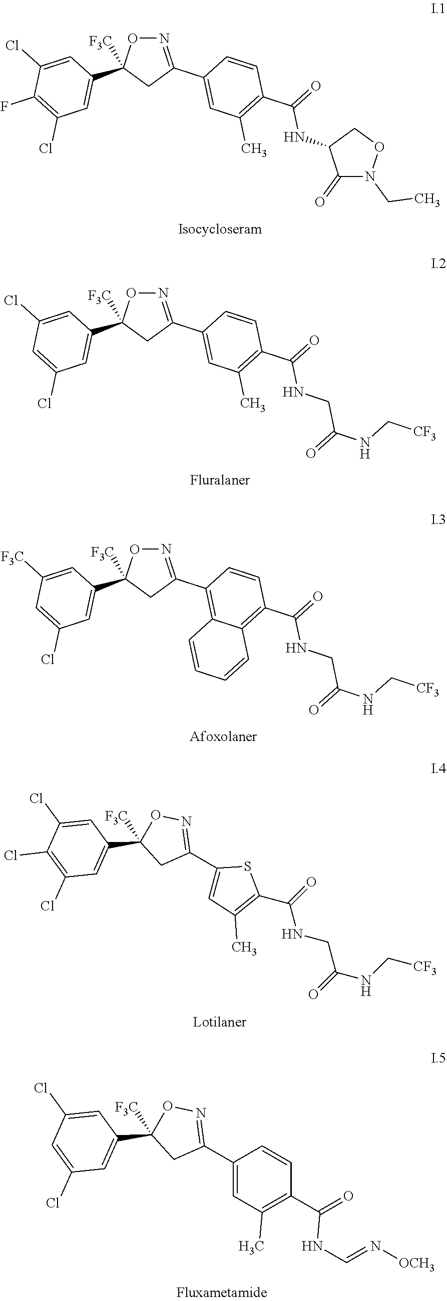 [SMILES: CCN1OC[C@@H](NC(=O)c2ccc(C3=NO[C@@](c4cc(Cl)c(F)c(Cl)c4)(C(F)(F)F)C3)cc2C)C1=O ; Cc1cc(C2=NO[C@@](c3cc(Cl)cc(Cl)c3)(C(F)(F)F)C2)ccc1C(=O)NCC(=O)NCC(F)(F)F ; O=C(CNC(=O)c1ccc(C2=NO[C@@](c3cc(Cl)cc(C(F)(F)F)c3)(C(F)(F)F)C2)c2ccccc12)NCC(F)(F)F ; Cc1cc(C2=NO[C@@](c3cc(Cl)c(Cl)c(Cl)c3)(C(F)(F)F)C2)sc1C(=O)NCC(=O)NCC(F)(F)F ; CO/N=C/NC(=O)c1ccc(C2=NO[C@@](c3cc(Cl)cc(Cl)c3)(C(F)(F)F)C2)cc1C]

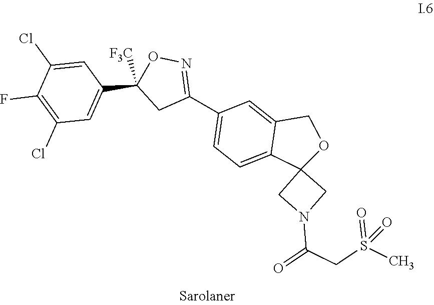 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

Accordingly, the process is furthermore particularly suitable for synthesis of compounds of formula I, wherein
R¹ is CF₃;
R^{2a} is F, Cl, Br, CF₃, or OCF₃;
R^{2b} and R^{2c} are independently from each other H, F, Cl, Br, CF₃, or OCF₃;
A is A¹, A², or A³; wherein
   A¹ is C(═O)N(R⁵)R⁶, C(═O)OR⁹, wherein
   A² is

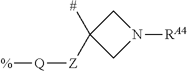

wherein # denotes the bond of group A, and % denotes the bond to G¹;
Q-Z is % —CH₂—O—*, wherein % marks the bond of Q to phenyl, and * the bond of Z to azetidin; and
R^{A4} is H, or C(═O)R^{4A}, wherein
R^{4A} is H, C₁-C₄-alkylcarbonyl, which is unsubstituted or substituted with S(O)ⁿ-C₁-C₆-alkyl;
A³ is CH₂—NR⁵C(═O)R⁶;
G¹, and G² are each CR³, or together form a sulfur atom;
   R³ is H or C₁-C₄-alkyl, or two R³ bonded to adjacent carbon atoms may form a five- or sixmembered saturated or aromatic carbocyclic ring, or a dihydrofurane, or
   R³ bonded to a carbon atom in position G¹ form a bond to the chain *-Q-Z- in group A²;
   R⁵ is H;
   R⁶ is H, or C₁-C₆-alkyl which is unsubstituted, or substituted with one or two R⁸;
   or R⁵ and R⁶, together with the nitrogen atom to which they are bound, form a 5- or 6-membered saturated, heterocyclic ring, which ring contain 1 or 2 groups selected from O, S, N, and C═O as ring members, which heterocyclic ring is unsubstituted or partially substituted with same or different C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl;
   each R⁸ is C(═O)N(R^{10a})R^{10b}, or
   two R⁸ present on the same carbon atom of an alkyl group together form ═NOR⁹;
   R⁹ being C₁-C₄-alkyl;
   R^{10a}, R^{10b} are independently from one another H, C₁-C₆-alkyl, C₁-C₆-haloalkyl, C₂-C₆-alkenyl, C₂-C₆-haloalkenyl, C₂-C₆-alkynyl, C₂-C₆-haloalkynyl.

Such compounds represent formula Ia.

Particularly preferred are intermediate compounds of formula Ia, which represents formula I wherein R¹ is CF₃, and the variables have the meanings as shown in Table I.1, wherein each compound corresponds to one line.

TABLE I.1

| No. | R^{2a}, R^{2b}, R^{2c} | 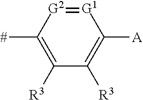 | A |
|---|---|---|---|
| Ia-1 | Cl, F, Cl | 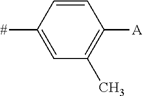 | COOH |
| Ia-2 | Cl, F, Cl | 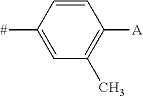 | COCH₃ |
| Ia-3 | Cl, F, Cl | 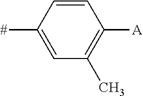 | COC₂H₅ |
| Ia-4 | Cl, F, Cl | 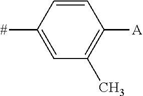 | CONH₂ |
| Ia-5 | Cl, F, Cl | 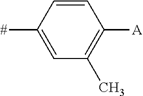 | Br |
| Ia-6 | Cl, F, Cl | 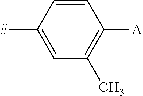 | I |
| Ia-7 | Cl, H, Cl | 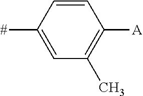 | COOH |
| Ia-8 | Cl, H, Cl | 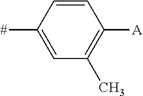 | COCH₃ |
| Ia-9 | Cl, H, Cl | 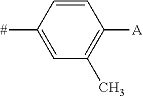 | COC₂H₅ |
| Ia-10 | Cl, H, Cl | 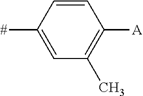 | CONH₂ |
| Ia-11 | Cl, H, Cl | 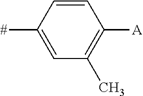 | Br |
| Ia-12 | Cl, H, Cl | 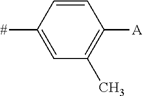 | I |
| Ia-13 | CF₃, H, Cl | 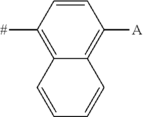 | COOH |
| Ia-14 | CF₃, H, Cl | 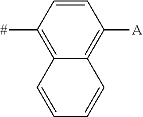 | COCH₃ |
| Ia-15 | CF₃, H, Cl | 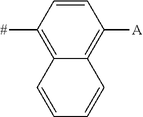 | COC₂H₅ |
| Ia-16 | CF₃, H, Cl | 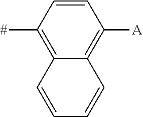 | CONH₂ |
| Ia-17 | CF₃, H, Cl | 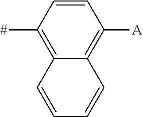 | Br |
| Ia-18 | CF₃, H, Cl | 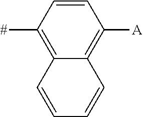 | I |
| Ia-19 | Cl, Cl, Cl | 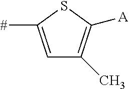 | COOH |
| Ia-20 | Cl, Cl, Cl | 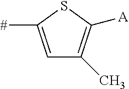 | COCH₃ |
| Ia-21 | Cl, Cl, Cl | 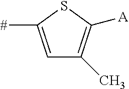 | COC₂H₅ |
| Ia-22 | Cl, Cl, Cl | 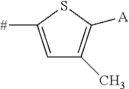 | CONH₂ |
| Ia-23 | Cl, Cl, Cl | 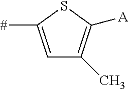 | Br |
| Ia-24 | Cl, Cl, Cl | 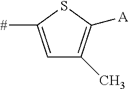 | I |

In the G¹-G² containing ring: # marks the bond to the isoxazoline group.

The following examples illustrate the invention.

### EXAMPLES

### A. Preparation Examples

With appropriate modification of the starting materials, the procedures given in the synthesis description were used to obtain further compounds I. The compounds obtained in this manner are listed in the table that follows, together with physical data.

The products shown below were characterized by melting point determination, by NMR spectroscopy or by the masses ([m/z]) or retention time (RT; [min.]) determined by HPLC-MS or HPLC spectrometry.

HPLC-MS=high performance liquid chromatography-coupled mass spectrometry;

HPLC method A: Shimadzu LC2010, Column: Waters XBridge C18, 150 mm*4.6 mm ID*5μ; Mobile Phase: A: water+0.1% TFA; B: acetonitrile+0.1% TFA; Temperature: 400° C.; Gradient: 10% B to 100% B in 5 min; 100% B 2 min; 10% B 3 min; Flow: 1.4 ml/min; Run Time: 10 min; PDA detector.

HPLC method B: Shimadzu LC2010, Column: CHIRALPAK AD-RH, 150 mm*4.6 mm*5μ; Mobile Phase: A: water+0.1% TFA; B: acetonitrile+0.1% TFA; Temperature: 400° C.; Gradient: 65% B to 100% B in 12 min; 100% B 1 min; 35% B 7 min; Flow: 1.4 ml/min; Run Time: 20 min; PDA detector.

### Example 1: Preparation of N-[[4-[(5S)-5-(3,5-dichloro-4-fluoro-phenyl)-5-(trifluoromethyl)-4H-isoxazol-3-yl]-2,3-dihydrobenzofuran-7-yl]methyl]propenamide

a) According to the invention with catalyst (R)-[1-[[10-[[2-[(R)-hydroxy-(6-methoxy-4-quinolyl)methyl]-5-vinyl-quinuclidin-1-ium-1-yl]methyl]-9-anthryl]methyl]-5-vinyl-quinuclidin-1-ium-2-yl]-(6-methoxy-4-quinolyl)methanol dibromide (III-Br₂)

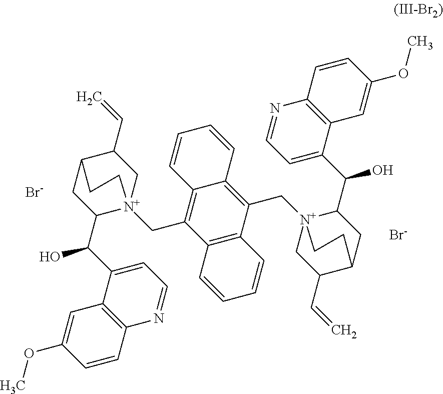

A round bottom glass flask was charged with 1 g (1 eq) of N-[[4-[(E)-3-(3,5-dichloro-4-fluoro-phenyl)-4,4,4-trifluoro-but-2-enoyl]-2,3-dihydrobenzofuran-7-yl]methyl]propenamide in 20 ml of DCE, the reaction mass was cooled to 0° C. and III-Br₂ 0.103 g (0.05 eq) was added. The reaction mass was stirred at 0° C. for 30 min. A premixed solution of 0.7 ml (5 eq) 50% NH₂OH solution in 2.5 ml (6 eq) of 20% NaOH was added dropwise in 90 mins and reaction mass was stirred at 0° C. for 5-6 hrs. After complete consumption of educts, water was added, and organic phase separated. Organic layer was washed with 6M HCl and water and evaporation of organic layer yielded 0.98 g (95.1% yield) of the title compound (99% HPLC purity, 92:8 S:R).

¹H-NMR (500 MHz, CDCl₃): 1.12-1.18 (t, 3H, J=7.5 Hz), 2.19-2.26 (q, 2H, J=7.8 Hz), 3.43-3.50 (m, 2H), 3.67-3.73 (dd, 1H, J=17 Hz), 4.06-4.12 (dd, 1H, J=17.1 Hz), 4.41-4.43 (d, 2 H, J=6 Hz), 4.63-4.69 (t, 2H, J=8.7 Hz), 6.03 (bs, 1H), 6.67-6.79 (d, 1H, J=7.8 Hz), 7.13-7.16 (d, 1H, J=7.8 Hz), 7.57-7.59 (d, 2H, 6Hz)

b) Comparison with (R)-[1-(9-anthrylmethyl)-5-vinyl-quinuclidin-1-ium-2-yl]-(6-methoxy-4-quinolyl)methanol chloride (PTC-1)

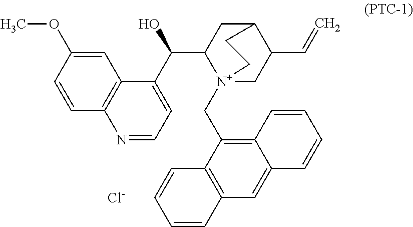 [SMILES: C=CC1C[N+]2(Cc3c4ccccc4cc4ccccc34)CCC1CC2[C@H](O)c1ccnc2ccc(OC)cc12 ; [Cl-]]

Analogously to Example 1, except replacement of the catalyst by 0.061 g (0.05 eq) of PTC-1 were obtained 0.75 g (72.8% yield) of the title compound (99.8% HPLC purity & 84:16 S:R).

### Example 2: Preparation of 4-[(5S)-5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide (isocycloseram, I.1)

Analogously to the protocol described in Example 1, isocycloseram was obtained

a) With III-Br₂: Enantiomeric ratio at isoxazoline=95:5 (S:R), 82% yield;

b) With PTC-1: Enantiomeric ratio at isoxazoline=81:19 (S:R), 81% yield.

1. A process for preparing an optically enriched isoxazoline compound of formula I 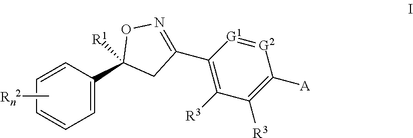
wherein
R¹ is halomethyl;
each R² is independently H, halogen, CN, N₃, NO₂, SCN, SF₅, C₁-C₆-alkyl, C₃-C₈-cycloalkyl, C₂-C₆-alkenyl, C₂-C₆-alkynyl, which groups are unsubstituted, partially or fully halogenated and/or substituted with one or more same or different R⁸,
Si(R¹²)₃, OR⁹, S(O)ₙR₉, NR^{10a}R^{10b},
phenyl which is unsubstituted or partially or fully substituted with R¹¹, and a 3- to 10-membered saturated, partially or fully unsaturated heteromonocyclic or heterobicyclic ring containing 1, 2, 3 or 4 heteroatoms N, O, and/or S as ring members, which ring is unsubstituted, or substituted with one or more same or different R¹¹;
n is 0, 1, or 2;
G¹, G² are each CR³, or together form a sulfur atom;
each R³ is independently selected from the meanings mentioned for R²,
or two R³ bonded to adjacent carbon atoms may form a five- or sixmembered saturated, partially or fully unsaturated carbocyclic ring, or a dihydrofurane, or
R³ bonded to carbon atom in position G¹ form a bond to the chain *-Q-Z- in group A²;
A is a group A¹, A², A³, or A⁴; wherein
A¹ is C(═W)Y;
W is O, or S;
Y is N(R⁵)R⁶, or OR⁹;
A² is 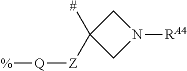
wherein # denotes the bond of group A, and % denotes the bond to G¹;
Q-Z is % —CH₂—O—*, ‘—CH₂—S(O)ₙ—*, or % —C(═O)—O—*, wherein % marks the bond of Q to phenyl, and * the bond of Z to azetidin; and
R^{A4} is H or C(═O)R^{4A}, wherein
R^{4A} is H, C, C₆ alkyl, C₁-C₆-haloalkyl, C₂-C₆-alkenyl, C₂-C₆-haloalkenyl, C₂-C₆-alkynyl, C₂-C₆-haloalkynyl, C₁-C₄-alkylcarbonyl, which aliphatic groups are unsubstituted or substituted with one or more radicals R⁴¹;
C₃-C₆-cycloalkyl, C₃-C₆-halocycloalkyl which cyclic groups are unsubstituted or substituted with one or more R⁴²;
C(═O)N(R⁴³)R⁴⁴, N(R⁴³)R⁴⁵, CH═NOR⁴⁶;
phenyl, heterocycle, or hetaryl which rings are unsubstituted or partially or fully substituted with R^{A};
R⁴¹ is independently OH, CN, C₁-C₆-alkoxy, C₁-C₆-haloalkoxy, S(O)ₙ-C₁-C₆-alkyl, S(O)ₙ-C₁-C₆-haloalkyl, C(═O)N(R⁴³)R⁴⁴,
C₃-C₆-cycloalkyl, or C₃-C₆-halocycloalkyl which cycles are unsubstitued or substituted with one or more R⁴¹¹; or
phenyl, heterocycle or hetaryl which rings are unsubstitued or partially or fully substituted with R^{A};
R⁴¹¹ is independently OH, CN, C₁-C₂-alkyl, or C₁-C₂-haloalkyl;
R⁴³ is H, or C₁-C₆-alkyl,
R⁴⁴ is H, C₁-C₆ alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted with a cyano;
R⁴⁵ H, C₁-C₆ alkyl, C₁-C₆-haloalkyl, C₂-C₄-alkenyl, C₂-C₄-alkynyl, CH₂—CN, C₃-C₆-cycloalkyl, C₃-C₆-halocycloalkyl, C₃-C₆-cycloalkylmethyl, C₃-C₆-halocycloalkylmethyl, phenyl and hetaryl which aromatic rings are unsubstituted or partially or fully substituted with R^{A};
R⁴² C₁-C₆-alkyl, C₁-C₆-haloalkyl, or a group as defined for R⁴¹;
R⁴⁶ is independently H, C₁-C₆-alkyl, or C₁-C₆-haloalkyl;
R^{A} is independently selected from halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₙ-C₁-C₄-alkyl, S(O)ₙ-C₁-C₄-haloalkyl, C₁-C₄-alkylcarbonyl, C₁-C₄-haloalkylcarbonyl, C(═O)N(R⁴³)R⁴⁴; or
two R^{A} present on the same carbon atom of a saturated or partially saturated ring may form together ═O or ═S; or
two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
A³ is CH₂—NR⁵C(═W)R⁶;
A⁴ is halogen, or cyano;
R⁵ is independently selected from the meanings mentioned for R²;
R⁶ is H, CN, C₁-C₁₀-alkyl, C₃-C₈-cycloalkyl, C₂-C₁₀-alkenyl, C₂-C₁₀-alkynyl, which groups are unsubstituted, partially or fully halogenated and/or substituted with one or more same or different R⁸; or
S(O)ₙR⁹, or C(═O)R⁸; or
a 3- to 8-membered saturated, partially or fully unsaturated heterocyclic ring, which ring may contain 1, 2, 3, or 4 heteroatoms O, S, N, C═O and/or C═S as ring members, which heterocyclic ring is unsubstituted or partially or fully substituted with same or different halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R⁸, or phenyl which may be partially or fully substituted with R¹¹;
or R⁵ and R⁶ together form a group ═C(R⁸)₂, ═S(O)ₘ(R⁹)₂, ═NR^{10a}, or ═NOR⁹;
R^{7a}, R^{7b} are each independently H, halogen, CN, C₁-C₆-alkyl, C₃-C₈-cycloalkyl, C₂-C₆-alkenyl, or C₂-C₆-alkynyl, which groups are unsubstituted, partially or fully halogenated and/or substituted with same or different R⁸;
each R⁸ is independently CN, N₃, NO₂, SCN, SF₅, C₃-C₈-cycloalkyl, C₃-C₈-halocycloalkyl, wherein the carbon chains may be substituted with one or more R¹³;
Si(R¹²)₃, OR⁹, OSO₂R⁹, S(O)ₙR⁹, N(R^{10a})R^{10b}, C(═O)N(R^{10a})R^{10b},
C(═S)N(R^{10a})R^{10b}, C(═O)OR⁹, CH═NOR⁹,
phenyl, which is unsubstituted or partially or fully substituted with same or different R¹⁶, or
a 3-, 4-, 5-, 6- or 7-membered saturated, partially or fully unsaturated heterocyclic ring comprising 1, 2 or 3 heteroatoms N, O, and/or S as ring members, which ring is unsubstituted or partially or fully substituted with same or different R¹⁶, or
two R⁸ present on the same carbon atom of an alkyl, alkenyl, alkynyl or cycloalkyl group together form a group ═O, ═C(R¹³)₂; ═S; ═S(O)ₘ(R¹⁵)₂,
═S(O)ₘR¹⁵N(R^{14a})R^{14b}, ═NR^{10a}, ═NOR⁹; or ═NN(R^{10a})R^{10b}; or
two radicals R⁸, together with the carbon atoms of the alkyl, alkenyl, alkynyl or cycloalkyl group which they are bonded to, form a 3-, 4-, 5-, 6-, 7- or 8-membered saturated or partially unsaturated carbocyclic or heterocyclic ring, which heterocyclic ring comprises 1, 2, 3 or 4 heteroatoms N, O, and/or S as ring members, and which ring is unsubstituted, or partially or fully substituted with same or different R¹⁶; and
R⁸ as a substituent on a cycloalkyl ring may additionally be C₁-C₆-alkyl, C₁-C₆-haloalkyl, C₂-C₆-alkenyl, C₂-C₆-haloalkenyl, C₂-C₆-alkynyl, and C₂-C₆-haloalkynyl, which groups are unsubstituted, or partially or fully substituted with same or different R¹³; and
R⁸ in the groups C(═O)R⁸ and ═C(R⁸)₂ may additionally be H, halogen, C₁-C₆-alkyl, C₁-C₆-haloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³;
each R⁹ is independently H, CN, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³, or
C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b}, C₁-C₆-alkyl-C(═NR¹⁴)N(R^{14a})R^{14b}, Si(R¹²)₃, S(O)ₙR¹⁵, S(O)ₙN(R^{14a})R^{14b}, N(R^{10a})R^{10b}, N═C(R¹³)₂, C(═O)R¹³, C(═O)N(R^{14a})R^{14b}, C(═S)N(R^{14a})R^{14b}, C(═O)OR¹⁵, or
phenyl, which is unsubstituted, or partially or fully substituted with R¹⁶; and
a 3- to 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; and
R⁹ in the groups S(O)ₙR⁹ and OSO₂R⁹ may additionally be C₁-C₆-alkoxy, or C₁-C₆-haloalkoxy;
R^{10a}, R^{10b} are independently from one another H, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R¹³;
C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b},
C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, S(O)ₙR¹⁵, S(O)ₙN(R^{14a})R^{14b}, C(═O)R¹³, C(═O)OR¹⁵, C(═O)N(R^{14a})R^{14b},
C(═S)R¹³, C(═S)SR¹⁵, C(═S)N(R^{14a})R^{14b}, C(═NR¹⁴)R¹³;
phenyl, which is unsubstituted, or partially or fully substituted with same or different R¹⁶; and
a 3-, 4-, 5-, 6- or 7-membered saturated, partially or fully unsaturated heterocyclic ring comprising 1, 2, 3 or 4 heteroatoms N, O, and/or S as ring members, which ring is unsubstituted, or partially or fully substituted with same or different R¹⁶, preferably unsubstituted or substituted hetaryl; or
R^{10a} and R^{10b} together with the nitrogen atom they are bonded to form a 3- to 8-membered saturated, partially or fully unsaturated heterocyclic ring, which ring may additionally contain one or two heteroatoms N, O, and/or S as ring members, which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with R¹⁶, and a 3-, 4-, 5-, 6,- or 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; or
R^{10a} and R^{10b} together form a group ═C(R¹³)₂, ═S(O)ₘ(R¹⁵)₂,
═S(O)ₘR¹⁵N(R^{14a})R^{14b}, ═NR¹⁴, or ═NOR¹⁵;
R¹¹ is halogen, CN, N₃, NO₂, SCN, SF₅, C₁-C₁₀-alkyl, C₃-C₈-cycloalkyl, C₂-C₁₀-alkenyl, C₂-C₁₀-alkynyl, which groups are unsubstituted, partially or fully halogenated, and/or may be substituted with same or different R⁸, or
OR⁹, NR^{10a}R^{10b}, S(O)ₙR⁹, Si(R¹²)₃;
phenyl, which is unsubstituted, or partially or fully substituted with same or different R¹⁶; and
a 3- to 7-membered saturated, partially or fully unsaturated aromatic heterocyclic ring comprising 1, 2, 3, or 4 heteroatoms N, O, and/or S as ring members, which ring is unsubstituted, or partially or fully substituted with same or different R¹⁶; or
two R¹¹ present on the same ring carbon atom of an unsaturated or partially unsaturated heterocyclic ring may together form a group ═O, ═C(R¹³)₂, ═S, ═S(O)ₘ(R¹⁵)₂, ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3- to 9-membered ring, which ring may contain 1 or 2 heteroatoms O, S, N, and/or NR¹⁴, and/or 1 or 2 groups C═O, C═S, C═NR¹⁴ as ring members, and which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with same or different R¹⁶, and a 3- to 7-membered saturated, partially or fully unsaturated heterocyclic ring containing 1, 2, or 3 heteroatoms N, O, and/or S as ring members, which ring is unsubstituted, or partially or fully substituted with same or different R¹⁶;
each R¹² is independently C₁-C₄-alkyl and phenyl, which is unsubstituted, or partially or fully substituted with same or different C₁-C₄-alkyl;
each R¹³ is independently CN, NO₂, OH, SH, SCN, SF₅, C₁-C₆-alkoxy, C₁-C₆-haloalkoxy, SOₙ-C₁-C₆-alkyl, SOₙ-C₁-C₆-haloalkyl, Si(R¹²)₃, —C(═O)N(R^{14a})R^{14b}, C₃-C₈-cycloalkyl which is unsubstituted, partially or fully halogenated or substituted with 1 or 2 same or different C₁-C₄-alkyl, C₃-C₄-cycloalkyl, C₁-C₄-alkoxy, C₁-C₄-haloalkoxy and/or oxo; phenyl, benzyl, phenoxy, where the phenyl moiety may be substituted with one or more same or different R¹⁶; and
a 3- to 7-membered saturated, partially or fully unsaturated heterocyclic ring containing 1, 2, or 3 heteroatoms N, O, and/or S, as ring members, which ring is unsubstituted, or partially or fully substituted with same or different R¹⁶; or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl); and
R¹³ as a substituent of a cycloalkyl ring may additionally be C₁-C₆-alkyl, C₂-C₆-alkenyl or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo; and
R¹³ in groups ═C(R¹³)₂, N═C(R¹³)₂, C(═O)R¹³, C(═S)R¹³, and C(═NR¹⁴)R¹³ may additionally be H, halogen, C₁-C₆-alkyl, C₂-C₆-alkenyl, or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo;
each R¹⁴ is independently H, CN, C₁-C₆-alkoxy, C₁-C₆-haloalkoxy, SOₙ-C₁-C₆-alkyl, SOₙ-C₁-C₆-haloalkyl, Si(R¹²)₃;
C₁-C₆-alkyl, C₂-C₆-alkenyl, C₂-C₆-alkynyl, which groups are unsubstituted, partially or fully halogenated, or substituted with 1 or 2 CN, C₁-C₄-alkoxy, C₁-C₄-haloalkoxy, SOₙ-C₁-C₄-alkyl, C₃-C₆-cycloalkyl which is unsubstituted or substituted with 1 or 2 substituents halogen and CN;
and oxo;
C₃-C₈-cycloalkyl which is unsubstituted, or partially or fully halogenated or substituted with 1 or 2 CN, C₁-C₄-alkyl, C₁-C₄-alkoxy, C₁-C₄-haloalkoxy, SOₙ-C₁-C₆-alkyl, C₃-C₄-cycloalkyl, C₃-C₄-cycloalkyl-C₁-C₄-alkyl-, which groups are unsubstituted, or substituted with 1 or 2 substituents selected from halogen and CN;
phenyl, benzyl, pyridyl, phenoxy, which cyclic moieties are unsubstituted, or substituted with one or more same or different halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and C₁-C₆-alkoxycarbonyl;
and a 3-, 4-, 5- or 6-membered saturated, partially or fully unsaturated heterocyclic ring comprising 1, 2 or 3 heteroatoms N, O, and/or S as ring members, which ring is unsubstituted, or partially or fully substituted with same or different R¹⁶;
R^{14a} and R^{14b}independently of each other, have one of the meanings given for R¹⁴; or
R^{14a} and R^{14b}, together with the nitrogen atom to which they are bound, form a 3- to 7-membered saturated, partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy; or
R^{14a} and R¹⁴ or R^{14b} and R¹⁴, together with the nitrogen atoms to which they are bound in the group C(═NR¹⁴)N(R^{14a})R^{14b}, form a 3- to 7-membered partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-haloalkyl, C₁-C₄-alkoxy, or C₁-C₄-haloalkoxy;
each R¹⁵ is independently H, CN, Si(R¹²)₃
C₁-C₆-alkyl, C₂-C₆-alkenyl, C₂-C₆-alkynyl, which groups are unsubstituted, partially or fully halogenated, or substituted with 1 or 2 radicals C₃-C₄-cycloalkyl, C₁-C₄-alkoxy, C₁-C₄-haloalkoxy, SOₙ-C₁-C₆-alkyl, or oxo;
C₃-C₈-cycloalkyl which is unsubstituted, partially or fully halogenated or substituted with 1 or 2 radicals C₁-C₄-alkyl, C₃-C₄-cycloalkyl, C₁-C₄-alkoxy, C₁-C₄-haloalkoxy, SOₙ-C₁-C₆-alkyl, or oxo;
phenyl, benzyl, pyridyl, and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl;
each R¹⁶ is independently halogen, NO₂, CN, OH, SH, C₁-C₆-alkoxy, C₁-C₆-haloalkoxy, SOₙ-C₁-C₆-alkyl, SOₙ-C₁-C₆-haloalkyl, C₁-C₄-alkylcarbonyl, C₁-C₄-haloalkylcarbonyl, C₁-C₄-alkoxycarbonyl, C₁-C₄-haloalkoxycarbonyl, aminocarbonyl, C₁-C₄-alkylaminocarbonyl, di-(C₁-C₄-alkyl)-aminocarbonyl, Si(R¹²)₃;
C₁-C₆-alkyl, C₂-C₆-alkenyl, C₂-C₆-alkynyl, which groups are unsubstituted, partially or fully halogenated, or substituted with 1 or 2 radicals CN, C₃-C₄-cycloalkyl, C₁-C₄-alkoxy, C₁-C₄-haloalkoxy, or oxo;
C₃-C₈-cycloalkyl which is unsubstituted, partially or fully halogenated or substituted with 1 or 2 radicals CN, C₁-C₄-alkyl, C₃-C₄-cycloalkyl, C₁-C₄-alkoxy, C₁-C₄-haloalkoxy, or oxo;
phenyl, benzyl, pyridyl and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl; or
two R¹⁶ present together on the same atom of an unsaturated or partially unsaturated ring may be ═O, ═S, ═N(C₁-C₆-alkyl), ═NO-C₁-C₆-alkyl, ═CH(C₁-C₄-alkyl), or ═C(C₁-C₄-alkyl)₂; or
two R¹⁶ on two adjacent carbon atoms form together with the carbon atoms they are bonded to a 4- to 8-membered saturated, partially or fully unsaturated ring, wherein the ring may contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy;
each n is independently 0, 1, or 2; and
each m is independently 0, or 1;
wherein the shown enantiomer has at least 80% ee;
comprising an oxo-Michael addition of hydroxyl amine or its salt to an enone of formula II, 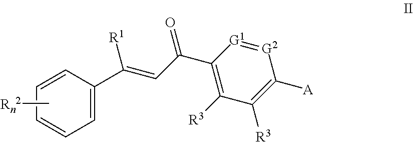
wherein the variables have the meanings given for formula I, in the presence of a catalyst of formula III 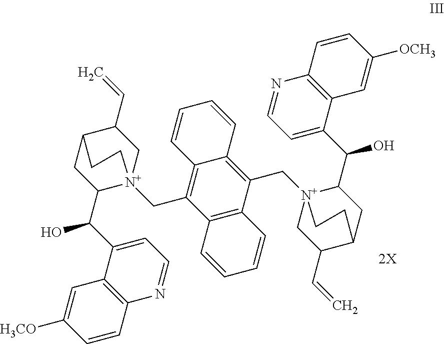
wherein X is a counteranion;
and a base.

2. The process according to claim 1, wherein group A in formula I is A¹.

3. The process according to claim 1, wherein group A in formula I is A².

4. The process according to claim 1, wherein group A in formula I is A³.

5. The process according to claim 1, wherein group A in formula I is A⁴.

6. The process according to claim 5, which further comprises reduction of I to a compound of formula Ic 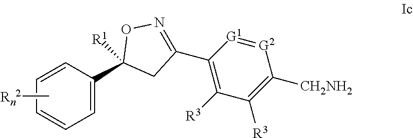
and acylation with a carboxylic acid derivative of formula V 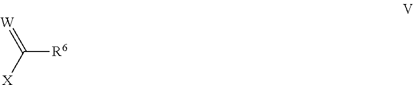
wherein the variables are as defined for formula I, 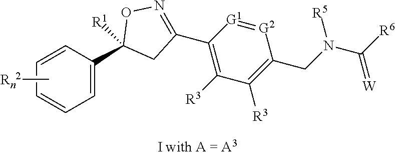
to yield a compound of formula I wherein A is A³.

7. The process according to claim 1, wherein the phenyl ring in formula I bearing the R²ₙ substitution is a group P 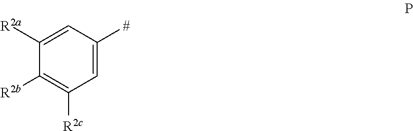
wherein R^{2a} is F, Cl, Br, CF₃, or OCF₃, and R^{2b} and R^{2c} are H, or as defined for R^{2a}.

8. The process according to claim 1, wherein R¹ is CF₃.

9. The process according to claim 1, wherein G¹ is C—CH₃, or C—Cl, and G² is CH.

10. The process according to claim 1, wherein in formula I group A is COOR⁹, wherein R⁹ is C₁-C₄-alkyl.

11. The process according to claim 1, wherein in formula I the variables have following meanings:
R¹ is CF₃;
R^{2a} is F, Cl, Br, CF₃, or OCF₃;
R^{2b} and R^{2c} are independently from each other H, F, Cl, Br, CF₃, or OCF₃;
A is A¹, A², or A³; wherein
A¹ is C(═O)N(R⁵)R⁶, C(═O)OR⁹, wherein
A² is 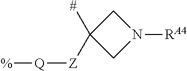
wherein # denotes the bond of group A, and % denotes the bond to G¹;
Q-Z is % —CH₂—O—*, wherein % marks the bond of Q to phenyl, and * the bond of Z to azetidin; and
R^{A4} is H, or C(═O)R^{4A}, wherein
R^{4A} is H, C₁-C₄-alkylcarbonyl, which is unsubstituted or substituted with S(O)ₙ-C₁-C₆-alkyl;
A³ is CH₂—NR⁵C(═O)R⁶;
G¹, and G² are each CR³, or together form a sulfur atom;
R³ is H or C₁-C₄-alkyl, or two R³ bonded to adjacent carbon atoms may form a five- or sixmembered saturated or aromatic carbocyclic ring, or a dihydrofurane, or
R³ bonded to a carbon atom in position G¹ form a bond to the chain *-Q-Z- in group A²;
R⁵ is H;
R⁶ is H, or C₁-C₆-alkyl which is unsubstituted, or substituted with one or two R⁸;
or R⁵ and R⁶, together with the nitrogen atom to which they are bound, form a 5- or 6-membered saturated, heterocyclic ring, which ring contain 1 or 2 groups selected from O, S, N, and C═O as ring members, which heterocyclic ring is unsubstituted or partially substituted with same or different C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl;
each R⁸ is C(═O)N(R^{10a})R^{10b}, or
two R⁸ present on the same carbon atom of an alkyl group together form ═NOR⁹;
R⁹ being C₁-C₄-alkyl;
R^{10a}, R^{10b} are independently from one another H, C₁-C₆-alkyl, C₁-C₆-haloalkyl, C₂-C₆-alkenyl, C₂-C₆-haloalkenyl, C₂-C₆-alkynyl, C₂-C₆-haloalkynyl.

12. The process according to claim 1, wherein formula I represents isocycloseram.

13. The process according to claim 1, wherein formula I is formula IA, 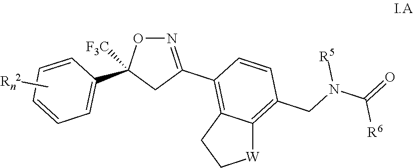
wherein R⁵ is H or CH₃, and R⁶ is H, C₁-C₆-alkyl, or C₂-C₆-alkenyl.

14. The process according to claim 1, wherein formula I is formula IB, 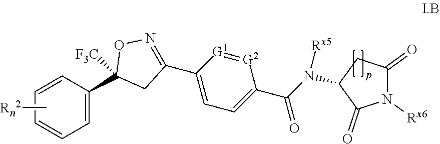
wherein
p is 1 or 2, R^{x5} is H or CH₃, and
R^{x6} is CH₃, C₂H₅, CH₂(CH₃)₂, CH₂CH═CH₂, CH₂CF₃, CH₂CH₂CF₃, CH₂C₆H₅, or CH₂C(═O)OCH₃.

15. The process according to claim 1, wherein the catalyst of formula III is applied in 0.01 to 0.5 mol equivalents of compound II.

16. The process according to claim 1, wherein an alkali metal or alkaline earth metal hydroxide is used as base in from 0.05 to 6 mol equivalents.

17. The process according to claim 1, wherein an amount of hydroxylamine is from 1 to 10 mol equivalents.
